(19) United States
(12) Patent Application Publication
(10) Pub. No.: US 2026/0283987 A1
(43) Pub. Date: Sep. 24, 2026

(54) HIGHLY WATER-DISPERSIBLE COMPOSITION HAVING HIGH CERAMIDE CONTENT

(71) Applicant: MOOGENE MEDI CO., LTD., Hanam-si, Gyeonggi-do (KR)

(72) Inventors: Kuk Youn JU, Hanam-si (KR); Kyeong Nam YU, Seoul (KR)

(73) Assignee: MOOGENE MEDI CO., LTD., Hanam-si, Gyeonggi-do (KR)

(21) Appl. No.: 19/471,894

(22) PCT Filed: Feb. 7, 2024
(86) PCT No.: PCT/KR2024/095092

§ 371 Date: Oct. 2, 2025

(30) Foreign Application Priority Data
Apr. 6, 2023 (KR) 10-2023-0045220

(51) Int. Cl. A61K 31/164; A61K 9/08; A61K 47/24; A61K 47/28

(52) U.S. Cl. CPC A61K 31/164; A61K 9/08; A61K 47/24; A61K 47/28

(57) ABSTRACT

Hydrophobic ceramides are of limited use due to having extremely low solubility in aqueous solutions, and have low cell transduction efficiency when used as a drug. The present invention provides a highly water-dispersible composition having a high ceramide content. A high dose of a hydrophobic ceramide can be easily delivered to a target in the body through the present invention to increase the utility as a highly water-dispersible drug carrier or pharmaceutical composition according to the intended purpose.

### TECHNICAL FIELD

The present disclosure relates to a highly water-dispersible ceramide composition capable of dispersing a hydrophobic ceramide in an aqueous solution without using any solvent such as alcohol, the ceramide composition being characterized by being dispersed in the aqueous solution at a solid content of up to 10 wt %.

### BACKGROUND ART

Ceramides are sphingolipids composed of sphingosine and fatty acids of various chain lengths. Generally, the endogenous forms of ceramides in animal cells are composed of various long-chain carbon lengths. In animal cells, six types of ceramide synthases exist, which serve to determine the acyl chain length of sphingolipids, including ceramides, sphingomyelins, and glycosphingolipids, by attaching fatty acyl-CoAs of specific lengths to long-chain bases.

Ceramides, categorized by the carbon chain length thereof, can act as active signaling molecules that regulate various cellular processes. Of all ceramides, hydrophobic long-chain ceramides, in particular, are synthesized by ceramide synthase 2. Ceramides, major components that constitute approximately 50% of the lipid layer in the skin tissue, serve to maintain the lipid barrier structure. Furthermore, ceramides, depending on the constituents thereof, are also known to act as anticancer agents by either promoting cell growth and proliferation or inducing cancer cell death. However, due to having extremely low solubility in aqueous solutions, these ceramides can only be applied by dissolution in organic solvents such as ethanol. For this reason, despite the strong cell proliferation and death efficacy of ceramides, the application thereof to various aqueous products is extremely limited.

In this respect, there is an urgent need for methods that enable long-chain ceramides having extremely low water solubility to be dispersed in water and the dispersed state to remain stable over a long period of time for practical use.

### DOCUMENTS OF RELATED ART

### Patent Documents

(Patent Document 1) Korean Patent No. 10-2325570 (Cosmetic composition for moisturizing and skin barrier improvement containing nanoliposome including oxygenated water and ceramide, and method of preparing same)
(Patent Document 2) Korean Patent No. 10-0713555 (Composition for hair protection containing, as active ingredient, nanoliposome stabilized by ceramide encapsulation)
(Patent Document 3) Korean Patent No. 10-2267378 (Pharmaceutical composition for preventing or treating breast cancer containing C12, C16, or C18 ceramide as active ingredient)
(Patent Document 4) Korean Patent No. 10-1796036 (Nanoliposome carrier composition encapsulating complex of Cas9 protein, guide RNA inhibiting expression of KRAS gene, and cationic polymer, or therapeutic for anticancer drug-resistant colorectal cancer containing same)
(Patent Document 5) Korean Patent No. 10-1710026 (Nanoliposome carrier composition containing hybrid of Cas9 protein and guide RNA
(Patent Document 6) Korean Patent No. 10-2328197 (Nanoliposome carrier composition having KRAS and p53 gene editing function)
(Patent Document 7) Japanese Patent No. 6944061 (Nanoliposome-microbubble conjugate encapsulating complex of Cas9 protein, guide RNA inhibiting expression of SRD5A2 gene, and cationic polymer, and composition for improving or treating hair loss containing same)
(Patent Document 8) Japanese Patent No. 6876202 (Nanoliposome-microbubble conjugate encapsulating drug for treating hair loss, and composition for improving or treating hair loss containing same)
(Patent Document 9) Japanese Patent No. 3920330 (Polyethylene glycol-modified ceramide lipids and use thereof as liposome)

### DISCLOSURE

### Technical Problem

The present disclosure aims to provide a highly water-dispersible ceramide composition capable of dispersing a hydrophobic ceramide in an aqueous solution without using any solvent such as alcohol, the ceramide composition being characterized by being dispersed in the aqueous solution at a solid content of up to 10 wt %.

### Technical Solution

The present disclosure relates to a composition having a water-dispersible formulation and including a hydrophobic ceramide dispersed in an aqueous solution, wherein the composition is characterized by including an anionic lipid, a neutral lipid, and cholesterol.

The hydrophobic ceramide may be included in the total composition in an amount of 40 mol % or more, but is preferably included in an amount of 60 mol % or less.

The anionic lipid may be included in the total composition in an amount in the range of 3 to 7 mol %.

The neutral lipid is characterized by being included in the total composition in an amount in the range of 7 to 10 mol %, and cholesterol is characterized by being included in the total composition in an amount in the range of 30 to 50 mol %.

The composition may be included in the aqueous solution at a solid content of 10 wt % or less.

The hydrophobic ceramide is characterized by having a structure of Formula 1 below.

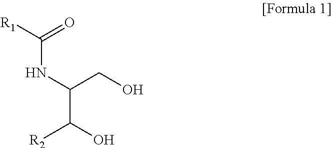

(In Formula 1, R₁ is a saturated or unsaturated aliphatic chain having 17 to 30 carbon atoms, and R₂ is a saturated or unsaturated aliphatic chain having 10 to 20 carbon atoms with or without a hydroxyl group.)

The neutral lipid may include one or more selected from the group consisting of hydrogenated soy phosphatidylcholine (HSPC), 1,2-dioleoyl-sn-glycero-3-phosphocholine (DOPC), 1-palmitoyl-2-oleoyl-sn-glycero-3-phosphoethanolamine (POPE), 1,2-distearoyl-sn-glycero-3-phosphocholine (DSPC), 1,2-distearoyl-sn-glycero-3-phosphoethanolamine (DSPE), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE), N-palmitoyl-D-erythro-sphingosylphosphorylcholine (SM), 1,2-dilauroyl-sn-glycero-3-phosphoethanolamine (DLPE), 1,2-diphytanoyl-sn-glycero-3-phosphoethanolamine (DPPE), phosphatidylcholine, dimyristoyl glycerol, succinoyl-diacylglycerol, phosphatidylethanolamine, tetraether lipids, sphingolipids, diacryl glycerol, and glyceride.

The anionic lipid is characterized by being a lipid including one or more selected from the group consisting of 1,2-distearoyl-sn-glycero-3-phosphatidylglycerol (DSPG), 1-stearoyl-2-oleoyl-sn-glycero-3-phosphatidylglycerol (SOPG), L-α-phosphatidylglycerol (Soy) (Soy PG), L-α-phosphatidylglycerol (Egg, Chicken) (Egg PG), 1,2-dilauroyl-sn-glycero-3-phosphatidylglycerol (DLPG), 1′,3′-bis [1,2-dioleoyl-sn-glycero-3-phospho]-glycerol (Cardiolipin), dimyristoylphosphatidylglycerol (DMPG), dipalmitoylphosphatidylglycerol (DPPG), dioleoylphosphatidylglycerol (DOPG), palmitoyloleoylphosphatidylglycerol (POPG), dioleoylphosphatidylserine (DOPS), diethylenetriamine pentaacetic acid (DTPA), 1,4-dipalmitoyl-tartrate-2,3-diglutaric acid (DPTGA), 1,4-distearoyl-tartrate-2,3-disuccinic acid (DSTSA), 2-carboxyheptadecanoyl heptadecylamide (CHHDA), dimyristoylphosphatidylserine (DMPS), dipalmitoylphosphatidylserine (DPPS), palmitoyloleoylphosphatidylserine (POPS), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

The ceramide composition may be a lipid nanoparticle, a nanoliposome, or an emulsion.

The hydrophobic ceramide having the structure of Formula 1 may be a compound selected from:
an N-stearoyl-erythro-sphingosine-based compound or a derivative thereof;
an N-oleoyl-erythro-sphingosine-based compound or a derivative thereof;
an N-stearoyl-erythro-sphinganine-based compound or a derivative thereof;
an N-oleoyl-erythro-sphinganine-based compound or a derivative thereof;
an N-nervonoyl-erythro-sphingosine-based compound or a derivative thereof;
an N-lignoceroyl-erythro-sphingosine-based compound or a derivative thereof;
an N-nervonoyl-erythro-sphinganine-based compound or a derivative thereof; and
an N-lignoceroyl-erythro-sphinganine-based compound or a derivative thereof.

The N-stearoyl-erythro-sphingosine-based compound or the derivative thereof may be
(E)-N-(1,3-dihydroxyoctadec-4-en-2-yl) stearamide or a derivative thereof, or
(E)-N-(1,3-dihydroxyheptadec-4-en-2-yl) stearamide or a derivative thereof.

The N-oleoyl-erythro-sphingosine-based compound or the derivative thereof may be
N-((E)-1,3-dihydroxyoctadec-4-en-2-yl) oleamide or a derivative thereof, or
N-((E)-1,3-dihydroxyheptadec-4-en-2-yl) oleamide or a derivative thereof.

The N-stearoyl-erythro-sphinganine-based compound or the derivative thereof may be
N-(1,3-dihydroxyoctadecan-2-yl) stearamide or a derivative thereof.

The N-oleoyl-erythro-sphinganine-based compound or the derivative thereof may be
N-(1,3-dihydroxyoctadecan-2-yl) oleamide or a derivative thereof.

The N-nervonoyl-erythro-sphingosine-based compound or the derivative thereof may be
(Z)-N-((4E,8Z)-1,3-dihydroxyoctadeca-4,8-dien-2-yl)tetracos-15-enamide or a derivative thereof,
(Z)-N-((E)-1,3-dihydroxyheptadec-4-en-2-yl)tetracos-15-enamide or a derivative thereof, or
(Z)-N-((E)-1,3-dihydroxyoctadec-4-en-2-yl)tetracos-15-enamide or a derivative thereof.

The N-lignoceroyl-erythro-sphingosine-based compound or the derivative thereof may be
N-((4E,8Z)-1,3-dihydroxyoctadeca-4,8-dien-2-yl)tetracosanamide or a derivative thereof, or
(E)-N-(1,3-dihydroxyoctadec-4-en-2-yl)tetracosanamide or a derivative thereof.

The N-nervonoyl-erythro-sphinganine-based compound or the derivative thereof may be
(Z)-N-(1,3-dihydroxyoctadecan-2-yl)tetracos-15-enamide or a derivative thereof.

The N-lignoceroyl-erythro-sphinganine-based compound or the derivative thereof may be
N-(1,3-dihydroxyoctadecan-2-yl)tetracosanamide or a derivative thereof.

The cholesterol imparts morphological rigidity to lipid packing in the lipid nanoparticle and is dispersed in the core and on the surface of the nanoparticle, thus serving to improve the stability of the nanoparticle.

The neutral lipid refers to a lipid that, as a nonionic surfactant, is uncharged or zwitterionic at a pH in the range of 4.0 to 8.0. In addition, the neutral lipid may include any neutral lipid known to those skilled in the art.

The anionic lipid refers to any amphipathic lipid that, as an anionic surfactant, carries one or more negative charges at a pH in the range of 4.0 to 8.0. In addition, the anionic lipid may include any anionic lipid known to those skilled in the art.

When the ceramide composition is the lipid nanoparticle, the lipid nanoparticle may have a particle size in the range of to 2,000 nm. When the size of the lipid nanoparticle falls below 10 nm or exceeds 2,000 nm, particle stability may be reduced, which is undesirable.

In the ceramide composition, the hydrophobic ceramide having the structure of Formula 1 is preferably included in an amount in the range of 40 to 60 mol % based on the total composition. Although the concentration of the hydrophobic ceramide may be lower than 40 mol %, the high-content ceramide is characterized by being included to improve the pharmacological and physiological efficacy that a drug carrier can exhibit. In addition, based on the total composition, when the anionic lipid is included in an amount of less than 3 mol % or more than 7 mol %, the neutral lipid is included in an amount of less than 7 mol % or more than 10 mol %, and the cholesterol is included in an amount of less than 30 mol % or more than 50 mol %, water dispersibility may fail to be maintained, or may appear stable for a short period of time, but the dispersed state after preparation may remain unstable for a long period of time, which is undesirable. In particular, the anionic lipid needs to be included in an amount of at least 3 mol % or more for the stability thereof.

The present disclosure also relates to a method of preparing the ceramide composition.

Preferably, a first step of preparing a mixed composition by mixing a hydrophobic ceramide of Formula 1 and one or more lipids selected from a neutral lipid, cholesterol, and an anionic lipid in an organic solvent, and
a second step of preparing the mixed composition in a dispersion state
are included.

In this case, in the first step, when the total amount of each raw material is 100 parts by weight, the organic solvent may be mixed in an amount in the range of 100 to 5000 parts by weight.

After the second step, the resulting dispersion may be subjected to high-pressure homogenization, as needed.

In addition, the dispersion may be subjected to column purification to obtain a purified product, followed by sterile filtration. The filter used for the sterile filtration is characterized by having a pore size in the range of 0.1 to 0.4 μm.

The high-pressure homogenization may be performed at a temperature in the range of 50° C. to 60° C. and a pressure in the range of 20000 to 30000 psi, and can be repeatedly performed one to three times. The pre-homogenization dispersion may be heated to a temperature in the range of 50° C. to 60° C. for use.

The organic solvent may be selected from methanol, ethanol, benzene, butanol, butyl acetate, carbon tetrachloride, chloroform, cyclohexane, dichloroethane, dichloromethane, diethyl ether, diisopropyl ether, ethyl acetate, heptane, hexane, isooctane, methyl ethyl ketone (MEK), methyl t-butyl ether (MTBE), pentane, toluene, trichloroethylene, xylene, and mixed solvents thereof.

In this case, in the second step, the dispersion may be prepared by sonication or freeze-thawing. Preferably, when a process of freezing or thawing the mixed composition is performed, this process is repeatedly performed one to 12 times. By repeatedly performing the steps of freezing and thawing the mixed composition, lipid nanoparticles of more uniform size can be formed, improving the formation efficiency of the lipid nanoparticles. Rather, evenly sized lipid nanoparticles may be less formed when repeatedly performing this process more than 12 times. For this reason, preferably, this process is repeatedly performed 12 times or fewer.

In this case, in the second step, the dispersion may be prepared in a separate reactor. The lipid nanoparticles may be formed by repeatedly performing the process of freezing and thawing the mixed composition in this reactor.

In another method, in the second step, the dispersion may be prepared by a droplet-based microfluidic system.

The composition of the present disclosure may be provided as a pharmaceutical composition or a cosmetic composition.

The pharmaceutical composition may be formulated and used in oral dosage forms, including powders, granules, tablets, capsules, suspensions, emulsions, syrups, and aerosols, as well as external preparations, suppositories, and sterile injections, according to respective conventional methods. Examples of carriers, excipients, and diluents that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oil. When being formulated, excipients or diluents, including commonly used fillers, bulking agents, binders, wetting agents, disintegrants, and surfactants, may be used for preparation. Solid preparations for oral administration may include tablets, pills, powders, granules, and capsules. Such solid preparations are prepared by mixing the composition of the present disclosure with at least one excipient, for example, starch, calcium carbonate, sucrose, lactose, or gelatin. In addition to simple excipients, lubricants such as magnesium stearate and talc are used. Examples of liquid preparations for oral administration include suspensions, oral liquids, and emulsions. In addition to simple diluents commonly used, such as water and liquid paraffin, syrups, various excipients, for example, wetting agents, sweetening agents, flavoring agents, and preservatives, may be included. Examples of preparations for parenteral administration include sterile aqueous solutions, non-aqueous solvents, suspensions, emulsions, lyophilized preparations, and suppositories. As for the non-aqueous solvents and suspensions, propylene glycol, polyethylene glycol, vegetable oils such as olive oil, injectable esters such as ethyl oleate, and the like may be used. As for suppository bases, Witepsol, macrogol, Tween 61, cacao fat, laurin fat, glycerogelatin, and the like may be used.

A dosage of the pharmaceutical composition of the present disclosure may vary depending on the age, sex, and weight of a subject to be treated, a specific disease or pathological condition to be treated, the severity of the disease or pathological condition, the administration route, and the decision of a prescriber. Dosage determination based on such factors falls within the level of those skilled in the art, and the dosage is generally in the range of 0.01 mg/kg/day to approximately 2000 mg/kg/day. More preferably, the dosage is in the range of 1 mg/kg/day to 500 mg/kg/day. The administration may be performed once or several times a day. The dosage is not intended to limit the scope of the present disclosure in any way.

Mammals, such as rats, livestock, and humans, may be administered the pharmaceutical composition of the present disclosure through various routes. While all administration methods are predictable, the pharmaceutical composition may be administered orally, rectally, or by injection, for example, intravenously, intramuscularly, subcutaneously, intrauterinely, intrathecally, or intracerebrovascularly.

The cosmetic composition of the present disclosure may be prepared in any formulations commonly prepared in the art, which may be provided as one selected from an essence, a lotion, an emulsion, a face mask, a hand cream, a foot cream, a lip balm, a lipstick, an eye shadow, an eyeliner, an eyebrow pencil, a blush, a highlighter, a general skin lotion, a toner, a cream, a serum, a beauty soap, a softening lotion, a medicated lotion, a body wash, a facial wash, a cleansing lotion, a gel, a cleansing oil, a cleansing cream, a shampoo, a conditioner, a hair treatment, a hair lotion, a cleansing wipe, and cleansing water.

The cosmetic composition may include any excipient composition commonly used. Preferably, the cosmetic composition may include oily components, emollients, surfactants, organic and inorganic pigments, organic powders, UV absorbers, antimicrobial agents, antioxidants, pH preservatives, adjusters, alcohols, colorants, fragrances, blood circulation promoters, cooling agents, antiperspirants, and purified water.

In addition, the cosmetic composition may include other active ingredients, which preferably include anti-wrinkle agents, anti-aging agents, lightening agents, antioxidants, anti-inflammatory agents, moisturizers, or hair growth agents.

As for the anti-wrinkle agent, substances inhibiting matrix metalloproteinase-1 (MMP-1), an enzyme that degrades extracellular matrix (ECM) proteins, may be selected, such as silicic acid, N-methyl-L-serine, isoflavonoids, dehydroepiandrosterone, and paeoniflorin. In addition, benzastatins, Coenzyme Q10, and the like may be used as substances preventing skin aging by removing active oxygen that promotes the breakdown of ECM. Furthermore, adenosine, ascorbyl glucoside, kinetin, auxin, peptides, retinol, retinyl palmitate, polyethoxylated retinamide, alpha-hydroxy acids, and the like, which are known for various anti-wrinkle effects, are available.

As for the lightening agent, arbutin, niacinamide, ascorbic acid, magnesium ascorbyl phosphate, ascorbic acid 2-glucoside, paper mulberry extract, ethyl ascorbyl ether, oil-soluble licorice extract, and the like are available.

As for the moisturizer, one or more selected from the group consisting of hydroxyproline, glycerin, glycerol, urea, amino acids, lactate, and pyroglutamic acid may be used.

The ceramide composition, when applied as a pharmaceutical composition or a cosmetic composition, may be used in a state including 1×10² to 1×10¹² particles per mL

### Advantageous Effects

Hydrophobic ceramides have been of limited use due to having extremely low solubility in aqueous solutions, and have exhibited low cellular uptake efficiency when used as drugs. Although existing technologies for converting ceramides into lipid nanoparticles have been confirmed, when such ceramides are included in an amount of more than 50% of the total lipid, the dispersion stability decreases and aggregation occurs, resulting in difficulties in biomedical applications.

On the contrary, the present disclosure provides a water-dispersed composition including a high content of a hydrophobic ceramide. Hence, the present disclosure enables the hydrophobic ceramide to be easily delivered to a target in the body, thereby increasing the utility thereof as a highly water-soluble and water-dispersible drug carrier or pharmaceutical composition according to the intended purpose.

### DESCRIPTION OF DRAWINGS

FIG. 1 illustrates a schematic diagram of a nanoparticle structure in a ceramide composition prepared in Example 1 of the present disclosure.

FIG. 2 shows images of ceramide compositions prepared using raw materials without anionic lipids, the images representing the states immediately after preparation.

FIG. 3 shows an image to confirm the respective states of nanoparticles, depending on the concentration of anionic lipids in the raw materials, after preparation of ceramide compositions.

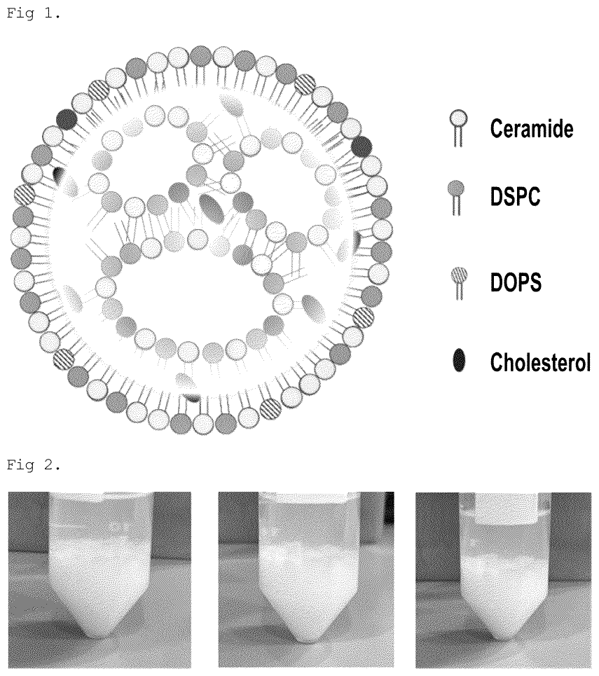

### MODE FOR INVENTION

Hereinafter, preferred embodiments of the present disclosure are to be described in detail. However, the present disclosure is not limited to the embodiments described herein and may be embodied in other forms. Rather, the embodiments are provided to sufficiently convey the idea of the present disclosure to those skilled in the art so that the description herein will be thorough and complete.

### Examples 1 to 12 and Comparative Examples 1 to 10

Compositions having a high ceramide content were prepared using a microfluidics.

First, the respective lipids were added to 1 mL of ethanol under the conditions of Table 1 below.

TABLE 1

| Lipid | Neutral lipid | | Ceramide | | Anionic lipid | | Cholesterol |
|---|---|---|---|---|---|---|---|
| nano- | | Concentration | | Concentration | | Concentration | Concentration |
| particle | Type | (mM) | Type | (mM) | Type | (mM) | (mM) |
| Example | DSPC | 8 | N- | 49 | DOPS | 5 | 38 |
| 1 | | | lignoceroyl- | | | | |
| | | | D-erythro- | | | | |
| | | | sphingosine | | | | |
| | | | (Merck | | | | |
| | | | #860524P) | | | | |
| Example | DSPC | 7 | N- | 40 | DOPS | 7 | 46 |
| 2 | | | lignoceroyl- | | | | |
| | | | D-erythro- | | | | |
| | | | sphingosine | | | | |
| | | | (Merck | | | | |
| | | | #860524P) | | | | |
| Example | DSPC | 7 | N- | 60 | DOPS | 3 | 30 |
| 3 | | | lignoceroyl- | | | | |
| | | | D-erythro- | | | | |
| | | | sphingosine | | | | |
| | | | (Merck | | | | |
| | | | #860524P) | | | | |
| Example | DSPC | 10 | N- | 50 | DOPS | 5 | 35 |
| 4 | | | lignoceroyl- | | | | |
| | | | D-erythro- | | | | |
| | | | sphingosine | | | | |
| | | | (Merck | | | | |
| | | | #860524P) | | | | |
| Example | DOPE | 10 | N- | 50 | DPPG | 5 | 35 |
| 5 | | | nervonoyl- | | | | |
| | | | D-erythro- | | | | |
| | | | sphingosine | | | | |
| | | | (Merck | | | | |
| | | | #860525P) | | | | |
| Example | DPPE | 10 | N- | 50 | DTPA | 5 | 35 |
| 6 | | | nervonoyl- | | | | |
| | | | D-erythro- | | | | |
| | | | sphingosine | | | | |
| | | | (Merck | | | | |
| | | | #860525P) | | | | |
| Example | DOPC | 10 | N- | 50 | POPS | 5 | 35 |
| 7 | | | nervonoyl- | | | | |
| | | | D-erythro- | | | | |
| | | | sphingosine | | | | |
| | | | (Merck | | | | |
| | | | #860525P) | | | | |
| Example | DPPC | 10 | N- | 50 | DMPS | 5 | 35 |
| 8 | | | nervonoyl- | | | | |
| | | | D-erythro- | | | | |
| | | | sphingosine | | | | |
| | | | (Merck | | | | |
| | | | #860525P) | | | | |
| Example | DSPC | 10 | N- | 50 | DOPS | 5 | 35 |
| 9 | | | nervonoyl- | | | | |
| | | | D-erythro- | | | | |
| | | | sphingosine | | | | |
| | | | (Merck | | | | |
| | | | #860525P) | | | | |
| Example | DSPC | 10 | N-oleoyl- | 50 | DOPS | 5 | 35 |
| 10 | | | D-erythro- | | | | |
| | | | sphingosine | | | | |
| | | | (Merck | | | | |
| | | | #860519P) | | | | |
| Example | DSPC | 10 | N- | 50 | DOPS | 5 | 35 |
| 11 | | | stearoyl- | | | | |
| | | | D-erythro- | | | | |
| | | | sphinganine | | | | |
| | | | (Merck | | | | |
| | | | #860627P) | | | | |
| Example | DSPC | 10 | N-oleoyl- | 50 | DOPS | 5 | 35 |
| 12 | | | D-erythro- | | | | |
| | | | sphinganine | | | | |
| | | | (Merck | | | | |
| | | | #860624P) | | | | |
| Comparative | DSPC | 12.5 | N- | 49 | DOPS | 0.5 | 38 |
| Example | | | lignoceroyl- | | | | |
| 1 | | | D-erythro- | | | | |
| | | | sphingosine | | | | |
| | | | (Merck | | | | |
| | | | #860524P) | | | | |
| Comparative | DSPC | 12 | N- | 49 | DOPS | 1 | 38 |
| Example | | | lignoceroyl- | | | | |
| 2 | | | D-erythro- | | | | |
| | | | sphingosine | | | | |
| | | | (Merck | | | | |
| | | | #860524P) | | | | |
| Comparative | DSPC | 10 | N- | 50 | DOPS | 0 | 40 |
| Example | | | lignoceroyl- | | | | |
| 3 | | | D-erythro- | | | | |
| | | | sphingosine | | | | |
| | | | (Merck | | | | |
| | | | #860524P) | | | | |
| Comparative | DSPC | 15 | N- | 20 | DOPS | 0 | 65 |
| Example | | | lignoceroyl- | | | | |
| 4 | | | D-erythro- | | | | |
| | | | sphingosine | | | | |
| | | | (Merck | | | | |
| | | | #860524P) | | | | |
| Comparative | DSPC | 0 | N- | 50 | DOPS | 5 | 45 |
| Example | | | lignoceroyl- | | | | |
| 5 | | | D-erythro- | | | | |
| | | | sphingosine | | | | |
| | | | (Merck | | | | |
| | | | #860524P) | | | | |
| Comparative | DSPC | 20 | N- | 40 | DOPS | 15 | 25 |
| Example | | | lignoceroyl- | | | | |
| 6 | | | D-erythro- | | | | |
| | | | sphingosine | | | | |
| | | | (Merck | | | | |
| | | | #860524P) | | | | |
| Comparative | DSPC | 15 | N- | 40 | DOPS | 10 | 35 |
| Example | | | lignoceroyl- | | | | |
| 7 | | | D-erythro- | | | | |
| | | | sphingosine | | | | |
| | | | (Merck | | | | |
| | | | #860524P) | | | | |
| Comparative | DSPC | 7 | N- | 40 | DOPS | 15 | 38 |
| Example | | | lignoceroyl- | | | | |
| 8 | | | D-erythro- | | | | |
| | | | sphingosine | | | | |
| | | | (Merck | | | | |
| | | | #860524P) | | | | |
| Comparative | DSPC | 5 | N- | 60 | DOPS | 10 | 25 |
| Example | | | lignoceroyl- | | | | |
| 9 | | | D-erythro- | | | | |
| | | | sphingosine | | | | |
| | | | (Merck | | | | |
| | | | #860524P) | | | | |
| Comparative | DOPC | 12.5 | N- | 49 | POPS | 0.5 | 38 |
| Example | | | nervonoyl- | | | | |
| 10 | | | D-erythro- | | | | |
| | | | sphingosine | | | | |
| | | | (Merck | | | | |
| | | | #860525P) | | | | |

Thereafter, to prepare the ceramide composition in a dispersion state, each raw material, once dissolved, was introduced into an organic solvent line, and water was introduced into a water solvent line, using microfluidic equipment, a droplet-based microfluidic system. In this case, the mixing ratio of the solvents was set to a 1:3 volume ratio of ethanol to water. After obtaining a liquid in a dispersion state in which nanoparticles were formed, ethanol was removed using a solvent replacement device. The ceramide composition in an ethanol-free dispersion state was subjected to homogenization twice using a high-pressure homogenizer at a temperature of 60° C. and a pressure of 27,000 psi, as needed. As a result, the ceramide composition in a dispersion state was obtained.

### Example 13

A composition having a high ceramide content was prepared using a thin-film hydration method as follows.

Four types of each lipid were added to 1 mL of chloroform. The types and total content ratios of each lipid and ceramide are the same as those in Example 1.

Thereafter, once each raw material was dissolved, the chloroform solvent was evaporated to form a lipid film. Next, the lipid film was hydrated with 1 mL of phosphate-buffered saline (PBS) (pH 7.2). After subjecting the hydrated liquid to sonication, a freeze-thaw procedure, in which the hydrated lipid film was repeatedly frozen and thawed using liquid nitrogen five times, was performed, followed by sonication again, thereby obtaining a liquid in a dispersion state. The resulting ceramide composition in a to dispersion state was subjected homogenization twice using a high-pressure homogenizer at a temperature of 60° C. and a pressure of 27,000 psi, as needed.

### <Experimental Example 1> Characterization of Compositions Having High Ceramide Content

The final particle size of the composition having a high ceramide content prepared in the present disclosure was measured, and dynamic light scattering (DIS) analysis was performed to show the measurements below. In addition, for each prepared ceramide composition, the number of nanoparticles prepared, as confirmed in the water-dispersed state, was approximately 1.0×10¹² to 6×10¹² particles/mL in the composition.

TABLE 2

| Ceramide composition | Number (particle size) (nm) |
|---|---|
| Example 1 | 74 ± 4.96 |
| Comparative Example 1 | 100 ± 3.00 |
| Comparative Example 2 | 171 ± 9.80 |

TABLE 3

| Ceramide composition | Z-Average (nm) |
|---|---|
| Example 1 | 122 ± 1.45 |
| Comparative Example 1 | 155 ± 2.00 |
| Comparative Example 2 | 208 ± 1.65 |

TABLE 4

| Ceramide composition | PDI (DLS) |
|---|---|
| Example 1 | 0.14 ± 0.01 |
| Comparative Example 1 | 0.12 ± 0.02 |
| Comparative Example 2 | 0.05 ± 0.01 |

TABLE 5

| | Water-dispersed formulation state | | | |
|---|---|---|---|---|
| | Immediately | 2 days | 7 days | 4 weeks |
| Ceramide | after | after | after | after |
| composition | preparation | preparation | preparation | preparation |
| Example 1 | ∘ | ∘ | ∘ | ∘ |
| Example 2 | ∘ | ∘ | ∘ | ∘ |
| Example 3 | ∘ | ∘ | ∘ | ∘ |
| Example 4 | ∘ | ∘ | ∘ | ∘ |
| Example 5 | ∘ | ∘ | ∘ | ∘ |
| Example 6 | ∘ | ∘ | ∘ | ∘ |
| Example 7 | ∘ | ∘ | ∘ | ∘ |
| Example 8 | ∘ | ∘ | ∘ | ∘ |
| Example 9 | ∘ | ∘ | ∘ | ∘ |
| Example 10 | ∘ | ∘ | ∘ | ∘ |
| Example 11 | ∘ | ∘ | ∘ | ∘ |
| Example 12 | ∘ | ∘ | ∘ | ∘ |
| Example 13 | ∘ | ∘ | ∘ | ∘ |
| Comparative | ∘ | x | — | — |
| Example 1 | | | | |
| Comparative | ∘ | x | — | — |
| Example 2 | | | | |
| Comparative | x | — | — | — |
| Example 3 | | | | |
| Comparative | x | — | — | — |
| Example 4 | | | | |
| Comparative | x | — | — | — |
| Example 5 | | | | |
| Comparative | ∘ | x | — | — |
| Example 6 | | | | |
| Comparative | ∘ | ∘ | x | — |
| Example 7 | | | | |
| Comparative | ∘ | ∘ | x | — |
| Example 8 | | | | |
| Comparative | ∘ | x | — | — |
| Example 9 | | | | |
| Comparative | ∘ | x | — | — |
| Example 10 | | | | |

∘: no abnormalities (stable state)
x: occurrence of abnormal phenomena such as aggregation
—: state no longer checked after occurrence of abnormal phenomena such as aggregation

The confirmation results showed that the physical properties of the particles did not differ significantly. Representatively, those in the case of Example 1 and Comparative Examples 1 and 2 are described and shown in Tables 2 to 4.

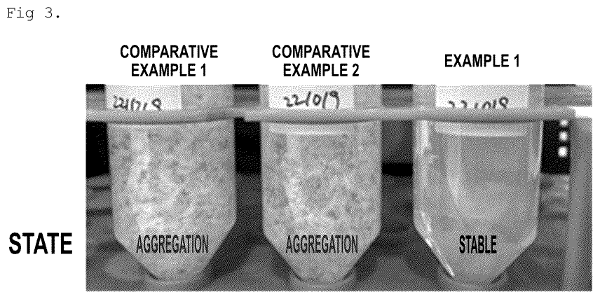

Regarding the confirmation results in terms of water dispersibility, the states of all the ceramide compositions prepared in the present disclosure are described in Table 5. Images for these results are shown representatively in FIGS. 2 and 3.

FIG. 2 shows images of the composition of Comparative Example 3, prepared without anionic lipids, the images being taken after filtration with a 0.2 μm syringe filter. Despite repeatedly conducting each experiment three times, the same state was observed. However, as shown in FIG. 2, when preparing the composition having a high ceramide content without anionic lipids, the dispersion stability of the composition in water decreased, resulting in aggregation and sedimentation. This composition is not re-dispersed even when performing additional high-pressure dispersion.

In the meantime, all the ceramide compositions prepared under the conditions of the examples of the present disclosure remained water-dispersed for four weeks or more, as shown in FIG. 3. FIG. 3 shows images of each composition stored for four weeks after performing the final high-pressure dispersion process. The compositions of Comparative Examples 1 and 2, containing a small amount of anionic lipids, appeared to have good water dispersibility immediately after preparation, but aggregation occurred on the second day, whereas the composition of Example 1 remained stable for up to four weeks.

As confirmed by the overall results of Table 5 and FIGS. 2 and 3, the physical properties of each particle appeared similar immediately after preparation. However, it was confirmed that only the ceramide composition prepared under the condition of the example of the present disclosure remained stable for four weeks or more.

### <Experimental Example 2> Confirmation of Cell Viability Following Treatment with Ceramide Composition

Dermal papilla (DP) cells, MDA-MB-231 cells, and MDA-MB-468 cells were cultured in American Type Culture Collection (ATCC)-modified Roswell Park Memorial Institute (RPMI) medium (Gibco) supplemented with 10% fetal bovine serum (FBS) and 1% penicillin/streptomycin, at a temperature of 37° C. in the presence of 5% CO₂.

For this experiment, the MDA-MB-231 and MDA-MB-468 cells inoculated into a 96-well plate (1.5×10³ cells/well) were treated with each of the ceramide compositions prepared in the present disclosure (number of particles: 4.5×10⁸±2.05×10⁸). After 24 hours of culture, the water-soluble tetrazolium salt-8 (WST-8, Dojindo) reagent was added to the medium at a temperature of 37° C. for 1 hour. Using a microplate reader (BioTek), absorbance was measured at a wavelength of 450 nm. Then, cell viability was confirmed by measuring absorbance every 24 hours.

As a result of the experiment, when the MDA-MB-231 cells were treated with the ceramide composition containing N-oleoyl-D-erythro-sphingosine (Merck), the cell viability decreased to an average of 50% to 70% upon treatment with this ceramide composition. In addition, when the MDA-MB-468 cells were treated with the ceramide composition containing N-oleoyl-D-erythro-sphingosine (Merck), the cell viability decreased to 40% to 55%.

Furthermore, when the DP cells were treated with the ceramide compositions containing N-lignoceroyl-D-erythro-sphingosine (Merck), the cell viability increased to an average of 300% to 400%. Even when the MDA-MB-468 cancer cells were treated with the ceramide compositions containing N-lignoceroyl-D-erythro-sphingosine (Merck) at various concentrations, there was no difference in cell viability, nor was any cytotoxicity observed.

In addition, for comparison with the ceramide compositions containing ceramides, ceramide alone was dispersed in an aqueous solution by itself at the same concentration used in the preparation of the ceramide compositions and then subjected to column purification to conduct a cell viability expression experiment under the same conditions as above. However, it was confirmed that there were no changes in cell death or proliferation. This is because the ceramide itself is insoluble in the aqueous solution and thus is absent as an active ingredient in the aqueous solution serving as the experimental sample. Accordingly, it is found that the effect of the ceramide itself was not observed.

These results demonstrate that the composition of the present disclosure has become a composition with high drug delivery efficiency by dispersing poorly soluble ceramide in water.

1. A highly water-dispersible ceramide composition being a composition having a water-dispersible formulation and comprising a hydrophobic ceramide dispersed in an aqueous solution,
wherein the composition comprises an anionic lipid, a neutral lipid, and cholesterol,
wherein the hydrophobic ceramide is included in the total composition in an amount of 40 mol % or more.

2. The composition of claim 1, wherein the anionic lipid is included in the total composition in an amount in a range of 3 to 7 mol %.

3. The composition of claim 1, wherein the composition is included in the aqueous solution at a solid content of 10 wt % or less.

4. The composition of claim 1, wherein the hydrophobic ceramide has a structure of Formula 1 below, 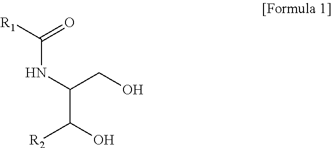
(in Formula 1, R₁ is a saturated or unsaturated aliphatic chain having 17 to 30 carbon atoms, and R₂ is a saturated or unsaturated aliphatic chain having 10 to 20 carbon atoms with or without a hydroxyl group).

5. The composition of claim 1, wherein the neutral lipid comprises one or more selected from the group consisting of hydrogenated soy phosphatidylcholine (HSPC), 1,2-dioleoyl-sn-glycero-3-phosphocholine (DOPC), 1-palmitoyl-2-oleoyl-sn-glycero-3-phosphoethanolamine (POPE), 1,2-distearoyl-sn-glycero-3-phosphocholine (DSPC), 1,2-distearoyl-sn-glycero-3-phosphoethanolamine (DSPE), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE), N-palmitoyl-D-erythro-sphingosylphosphorylcholine (SM), 1,2-dilauroyl-sn-glycero-3-phosphoethanolamine (DLPE), 1,2-diphytanoyl-sn-glycero-3-phosphoethanolamine (DPPE), phosphatidylcholine, dimyristoyl glycerol, succinoyl-diacylglycerol, phosphatidylethanolamine, tetraether lipids, sphingolipids, diacryl glycerol, and glyceride.

6. The composition of claim 1, wherein the anionic lipid is a lipid comprising one or more selected from the group consisting of 1,2-distearoyl-sn-glycero-3-phosphatidylglycerol (DSPG), 1-stearoyl-2-oleoyl-sn-glycero-3-phosphatidylglycerol (SOPG), L-α-phosphatidylglycerol (Soy) (Soy PG), L-α-phosphatidylglycerol (Egg, Chicken) (Egg PG), 1,2-dilauroyl-sn-glycero-3-phosphatidylglycerol (DLPG), 1′,3′-bis [1,2-dioleoyl-sn-glycero-3-phospho]-glycerol (Cardiolipin), dimyristoylphosphatidylglycerol (DMPG), dipalmitoylphosphatidylglycerol (DPPG), dioleoylphosphatidylglycerol (DOPG), palmitoyloleoylphosphatidylglycerol (POPG), dioleoylphosphatidylserine (DOPS), diethylenetriamine pentaacetic acid (DTPA), 1,4-dipalmitoyl-tartrate-2,3-diglutaric acid (DPTGA), 1,4-distearoyl-tartrate-2,3-disuccinic acid (DSTSA), 2-carboxyheptadecanoyl heptadecylamide (CHHDA), dimyristoylphosphatidylserine (DMPS), dipalmitoylphosphatidylserine (DPPS), palmitoyloleoylphosphatidylserine (POPS), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

7. The composition of claim 1, wherein the composition is a lipid nanoparticle.

8. The composition of claim 1, wherein the composition is a nanoliposome.

9. The composition of claim 1, wherein the composition is an emulsion.
